(19) United States
(12) Patent Application Publication
(10) Pub. No.: US 2025/0072970 A1
(43) Pub. Date: Mar. 6, 2025

(54) SYSTEMS AND METHODS FOR GUIDING A BLOOD FLOW RESTORATION PROCEDURE

(71) Applicant: Covidien LP, Mansfield, MA (US)

(72) Inventor: Varun Umesh Kashyap, Palo Alto, CA (US)

(21) Appl. No.: 18/821,280

(22) Filed: Aug. 30, 2024

Related U.S. Application Data
(60) Provisional application No. 63/579,874, filed on Aug. 31, 2023.

(51) Int. Cl. A61B 34/10; A61B 17/221; G06T 7/00; G06T 7/246; G06T 7/73

(52) U.S. Cl. CPC A61B 34/10; A61B 17/221; G06T 7/0016; G06T 7/248; G06T 7/74; A61B 2017/2215; A61B 2034/104; A61B 2034/105; G06T 2207/20081; G06T 2207/30104; G06T 2207/30204

(57) ABSTRACT

Systems and methods for guiding a blood flow restoration procedure (e.g., thrombectomy) are disclosed herein. In some variations in accordance with the present technology, a method for guiding removal of a thrombus includes receiving image data indicative of positions of marker elements on a thrombectomy device positioned proximate the thrombus in a patient, and predicting at least one characteristic of the thrombus by applying a trained machine learning algorithm to the image data.

### CROSS-REFERENCE TO RELATED APPLICATIONS

This application claims the benefit of and priority to U.S. Provisional Patent Application Ser. No. 63/579,874 filed Aug. 31, 2023, the entire disclosure of which is incorporated by reference herein.

### TECHNICAL FIELD

The present technology relates to systems and methods for guiding thrombectomy and/or other blood flow restoration procedures.

### BACKGROUND

Occlusion of blood vessels, such as by thrombi (e.g., clots), can be problematic and lead to significant adverse effects. For example, acute ischemic stroke (AIS) is a type of stroke where a blood clot occludes a vessel, causing decreased blood flow to the brain and resulting in damage to brain cells due to insufficient oxygen and nutrients being delivered to tissues downstream of the occlusion. Such damage to brain cells can, in many instances, leads to long-term disability. Mechanical thrombectomy has become a standard of care to treat patients with AIS. In this procedure, a stent retriever is delivered to the occlusion site (e.g., through the thrombus) and mechanically engages with the thrombus due at least in part to its outward radial force. Once engaged with the thrombus, the stent retriever can be withdrawn to retrieve the clot from the vasculature, thereby restoring blood flow in the formerly occluded vessel.

Mechanical thrombectomy is typically performed under fluoroscopy. However, since a thrombus is not radiopaque, thrombectomy is performed without being able to directly visualize the thrombus. While one technique to visualize the thrombus involves injecting a radiopaque substantive into the clot itself, this technique is not reliably effective and may cause further fragmentation of the clot, thereby leading to additional complications such as additional occlusion(s) to blood flow.

Inability to visualize the thrombus leads to potential challenges during thrombus retrieval and reduced retrieval efficacy. For example, currently, multiple thrombus retrieval cycles are often needed to retrieve the entire thrombus from the blood vessel, as the first pass efficacy (the percentage of time that the entire thrombus is retrieved in the first attempt) is only about 35%-40%. However, it is desirable to improve the first pass efficacy, since stroke is a time-sensitive disease state and brain cells continue dying every second blood flow is not restored. Furthermore, even if the entire thrombus is initially engaged by the stent retriever, parts of the thrombus may potentially break off from the stent retriever during withdrawal of the stent retriever, and such parts of the thrombus remain unseen until subsequent assessment shows additional occlusion(s) to blood flow.

Thus, there is a need for new and improved systems and methods for guiding thrombectomy.

### SUMMARY

The present technology is illustrated, for example, according to various aspects described below, including with reference to FIGS. 1-10. Various examples of aspects of the present technology are described as numbered examples (1, 2, 3, etc.) for convenience. These are provided as examples and do not limit the subject technology.

Example 1. A method for guiding removal of a thrombus, the method comprising: receiving image data indicative of positions of marker elements on a thrombectomy device positioned proximate the thrombus in a patient; and predicting at least one characteristic of the thrombus by applying a trained machine learning algorithm to the image data.

Example 2. The method of example 1, wherein the machine learning algorithm analyzes three-dimensional positions of the marker elements.

Example 3. The method of example 1 or 2, wherein the machine learning algorithm analyzes the position of a marker element at a given timepoint.

Example 4. The method of any one of examples 1-3, wherein the machine learning algorithm analyzes a change in position of a marker element across a period of time.

Example 5. The method of any one of examples 1-4, wherein the machine learning algorithm analyzes the relative positions of multiple marker elements at a given timepoint.

Example 6. The method of any one of examples 1-5, wherein the machine learning algorithm analyzes a change in the relative positions of multiple marker elements across a period of time.

Example 7. The method of any one of examples 1-6, wherein predicting at least one characteristic of the thrombus comprises predicting a boundary of the thrombus.

Example 8. The method of example 7, wherein predicting at least one characteristic of the thrombus comprises predicting the boundary of the thrombus during expansion of the thrombectomy device in the patient.

Example 9. The method of example 7 or 8, wherein predicting at least one characteristic of the thrombus comprises predicting the boundary of the thrombus after expansion of the thrombectomy device in the patient.

Example 10. The method of any one of examples 7-9, wherein predicting at least one characteristic of the thrombus comprises predicting the boundary of the thrombus during withdrawal of the thrombectomy device from the patient.

Example 11. The method of any one of examples 1-10, wherein predicting at least one characteristic of the thrombus comprises predicting a material characteristic of the thrombus.

Example 12. The method of example 11, wherein the material characteristic of the thrombus comprises stiffness of the thrombus.

Example 13. The method of any one of examples 1-12, wherein the machine learning algorithm analyzes a change in relative positions of the marker elements.

Example 14. The method of any one of examples 1-13, further comprising providing the predicted characteristic of the thrombus to a user.

Example 15. The method of example 14, wherein providing the predicted characteristic of the thrombus comprises displaying the predicted characteristic of the thrombus on a display screen.

Example 16. The method of any one of examples 1-15, wherein the thrombectomy device comprises a stent retriever.

Example 17. The method of any one of examples 1-16, wherein the marker elements are distributed around a circumference of the thrombectomy device.

Example 18. The method of any one of examples 1-17, wherein the marker elements are distributed along an axial length of the thrombectomy device.

Example 19. The method of any one of examples 1-18, wherein the marker elements comprise radiopaque elements.

Example 20. A system comprising: one or more processors; and a memory operably coupled to the one or more processors and storing instructions that, when executed by the one or more processors, cause the computing system to perform operations comprising the method of any one of examples 1-19.

Example 21. The system of example 20, wherein the memory is part of an angiography imaging system.

Example 22. A non-transitory computer-readable storage medium comprising instructions that, when executed by one or more processors of a computing system, cause the computing system to perform operations comprising the method of any one of examples 1-19.

Example 23. A method of treatment, comprising:

receiving at least one predicted characteristic of the thrombus predicted by the method of any one of examples 1-19.

Example 24. The method of example 23, wherein the at least one predicted characteristic of the thrombus comprises a boundary of the thrombus during deployment of the thrombectomy device, wherein the method further comprises adjusting position of the thrombectomy device based at least in part on the predicted boundary of the thrombus during deployment of the thrombectomy device.

Example 25. The method of example claim 23 or 24, wherein the at least one predicted characteristic of the thrombus comprises a boundary of the thrombus after deployment of the thrombectomy device, wherein the method further comprises confirming engagement of the thrombectomy device with the thrombus based at least in part on the predicted boundary of the thrombus after deployment of the thrombectomy device.

Example 26. The method of any one of examples 23-25, wherein the at least one predicted characteristic of the thrombus comprises a boundary of the thrombus during withdrawal of the thrombectomy device from the patient, wherein the method further comprises adjusting a technique of the withdrawal of the thrombectomy device based at least in part on the predicted boundary of the thrombus during withdrawal of the thrombectomy device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a schematic illustration of an example variation of a thrombectomy device, in accordance with the present technology.

FIG. 1B is a schematic illustration of an example variation of a thrombectomy device in an unrolled form, in accordance with the present technology.

FIG. 2 is a schematic illustration of an example variation of a thrombectomy device, in accordance with the present technology.

FIG. 3A is a flowchart depicting processes of an example variation of a method for guiding a blood flow restoration procedure, in accordance with the present technology.

FIG. 3B is a schematic illustration of a guidance system in relation to a blood flow restoration procedure, in accordance with the present technology.

FIG. 3C in a schematic illustration of a guidance system for a blood flow restoration procedure, in accordance with the present technology.

FIGS. 4, 5, 6A, 7, 8A, 9A, and 10 are schematic illustrations of various portions of a blood flow restoration procedure, in accordance with the present technology.

FIGS. 6B, 8B, and 9B are a schematic representation of fluoroscopic images of a thrombectomy device during various portions of a blood flow restoration procedure, in accordance with the present technology.

FIGS. 8C and 9C are schematic representations of images of a thrombectomy device overlaid or combined with predicted characteristic(s) of a thrombus during various portions of a blood flow restoration procedure, in accordance with the present technology.

### DETAILED DESCRIPTION

The present technology relates to systems and methods for guiding a blood flow restoration procedure. Some variations of the present technology, for example, are directed to treatment of acute ischemic stroke with mechanical thrombectomy guided with one or more trained machine learning algorithms. However, the present technology can additionally or alternatively be used for other blood flow restoration procedures where visualization of a thrombus may be advantageous.

For example, described herein are systems and methods for guiding thrombectomy using one or more trained machine learning algorithms. The machine learning algorithm can be configured to analyze image data (e.g., fluoroscopic images) that shows or otherwise is indicative of relative positions of marker elements on a thrombectomy device that is positioned proximate a thrombus in a patient. For example, the thrombectomy device can be positioned adjacent to the thrombus, such as partially or wholly engaged with the thrombus. Through its analysis, the machine learning algorithm can predict at least one characteristic of the thrombus, such as a boundary of the thrombus and/or a material characteristic of the thrombus (e.g., stiffness). A prediction of the thrombus boundary can provide information regarding the position of the thrombus with respect to the thrombectomy device, while a prediction of the stiffness of the thrombus can provide information regarding the mechanical stability of the thrombus (which can be useful because softer thrombi can often be more easily engaged with a thrombectomy device, while stiffer thrombi can often be more challenging to engage with a thrombectomy device).

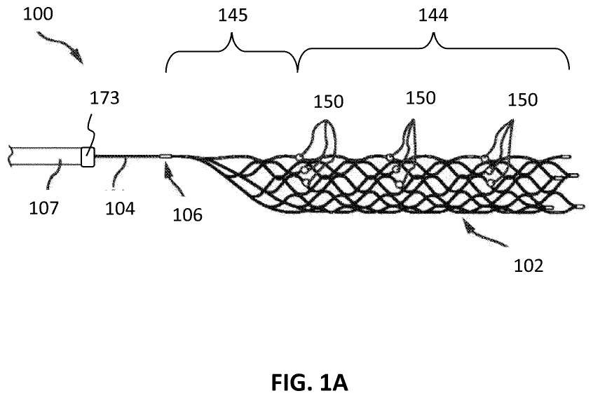

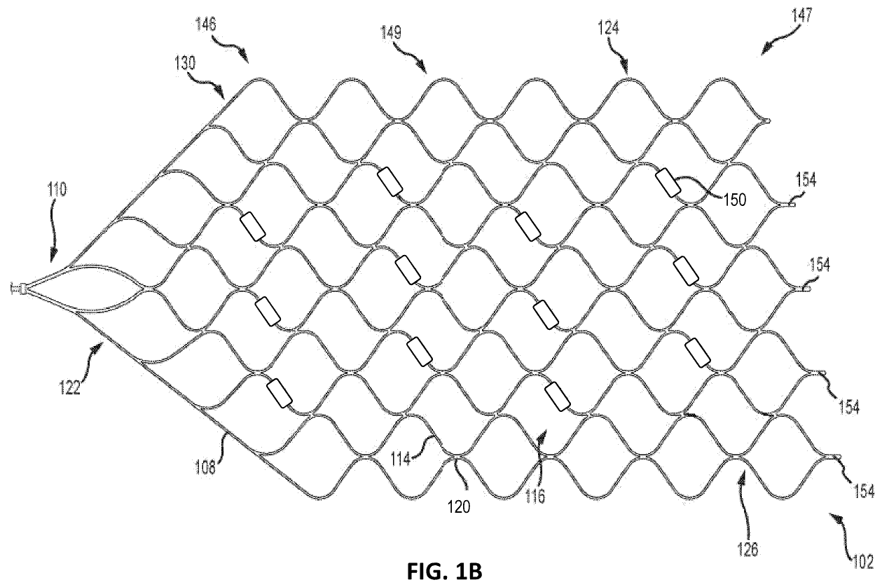

Accordingly, in some variations, the machine learning algorithm can be used to visualize and/or otherwise characterize the thrombus (e.g., without requiring injection of a radiopaque substance into the thrombus) during a thrombectomy procedure. This characterization of the thrombus, which may be referred to in some instances as a “digital clot twin” can be communicated to a user of the thrombectomy device (e.g., clinician), and the user can make adjustments to the thrombectomy procedure to improve procedure outcome, such as increasing the likelihood of retrieving the entire thrombus on the first retrieval attempt. Specific details of several variations of the present technology are described herein with reference to FIGS. 1-10.

### I. Thrombectomy System

As shown in FIG. 1A, a medical device 100 (e.g., thrombectomy system) can include a manipulation member 104 and a vessel device or thrombectomy device 102. A proximal end portion of the thrombectomy device 102 can be coupled to the distal end portion of the manipulation member 104, such as via a connection 106. FIG. 1A also shows a catheter 107 that can be navigated through vasculature of a patient toward a target treatment site (e.g., occlusion in a blood vessel caused by a thrombus), such as with a guidewire (not shown). The catheter 107 can, in some variations, include one or more markers 173, such as at a distal end of the catheter 107. The marker(s) 173 can include a radiopaque material such as platinum, iridium, tantalum, gold, alloys thereof or bismuth and/or tungsten-doped polymers, among other materials. The marker 173 can be visible under fluoroscopy, CT, X-rays, MRI, ultrasound, and/or other type(s) of imaging.

The manipulation member 104 can extend through a lumen of the catheter 107, such that an operator can manipulate the thrombectomy device 102, positioned within and/or distal to a distal end of the catheter 107, using the manipulation member 104 at a location near a proximal end of the catheter 107. For example, the thrombectomy device 102 can be delivered in a collapsed or delivery configuration to a target treatment site via the manipulation member 104 and/or the catheter 107, and the catheter 107 can be withdrawn proximally to at least partially expose at least the thrombectomy device 102 at or near the target treatment site. The exposed thrombectomy device 102 can expand to an expanded configuration (e.g., self-expand and/or expand with the aid of an expansion member such as a balloon) to engage a thrombus at the target treatment site.

The manipulation member 104 can be an elongate manipulation member. The manipulation member 104 can have a length sufficient to extend from a location outside the patient's body through the vasculature to a treatment site within the patient's body. For example, the manipulation member can have a length of at least 100 cm, at least 130 cm, or at least 150 cm. The manipulation member 104 can be monolithic or formed of multiple joined components. In some embodiments, the manipulation member 104 can comprise a combination of wire(s), coil(s), and/or tube(s).

The thrombectomy device 102 and the manipulation member 104 can be attached together at the connection 106. In some embodiments, the thrombectomy device 102 and the manipulation member 104 can be substantially permanently attached together at the connection 106. That is, the thrombectomy device 102 and the manipulation member 104 can be attached together in a manner such that, under the expected use conditions of the medical device 100, the thrombectomy device and the manipulation member would not become separated, whether deliberately or unintentionally, from one another without damage to or destruction of at least a portion of the connection 106. In some embodiments, the thrombectomy device 102 and the manipulation member 104 can be permanently or releasably attached together at the connection 106. In other variations, the thrombectomy device 102 can be integrally formed with the manipulation member 104.

In some variations, the connection 106 can include at least one marker. The marker of the connection 106 can include a radiopaque material such as platinum, iridium, tantalum, gold, alloys thereof or bismuth and/or tungsten-doped polymers, among other materials. The connection marker can be more radiopaque than a body of the thrombectomy device 102. The marker can be visible under fluoroscopy, CT, X-rays, MRI, ultrasound, and/or other type(s) of imaging. In some variations, the connection marker can include an interior channel, an interior recess or another mounting feature. For example, the connection marker can include a band or substantially cylindrical shape with an open or closed circumference, a coil, or other form.

In some variations, the connection 106 may be configured to permit intentional release of the thrombectomy device 102 from the manipulation member 104. For example, it may be difficult and/or dangerous to fully retrieve a thrombus in a thrombectomy procedure, such as due to a complicated vasculature or the risk of damage to a lumen wall. For these and other suitable situations, the thrombectomy device 102 may include drug-eluting capabilities, and/or may be coated with a particular type of drug that facilitates thrombus dissolution, as it may be advantageous in certain circumstances to release the thrombectomy device 102 and allow the drug-eluting or drug-coated thrombectomy device 102 to anchor the thrombus against the lumen wall while the thrombus is dissolved by the drug. In some variations, the medical device 100 can include a portion located proximally or distally of the connection 106, that is configured for selective detachment of the thrombectomy device 102 from the manipulation member 104. For example, such a portion can comprise an electrolytically severable or mechanically detachable segment of the manipulation member. In some variations, the medical device 100 can lack feature(s) that would permit selective detachment of the thrombectomy device 102 from the manipulation member 104.

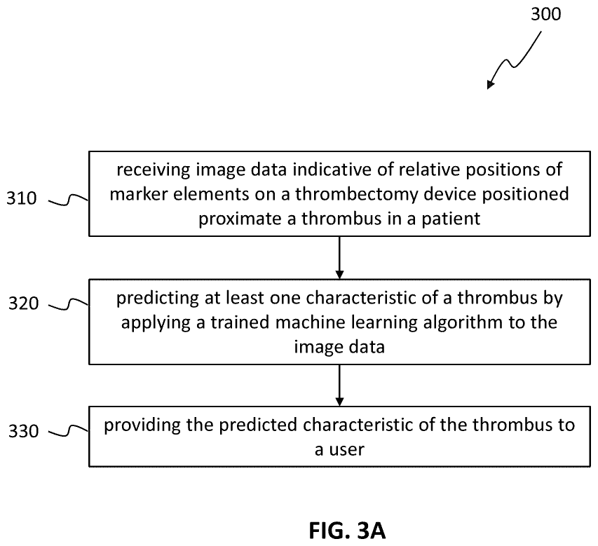

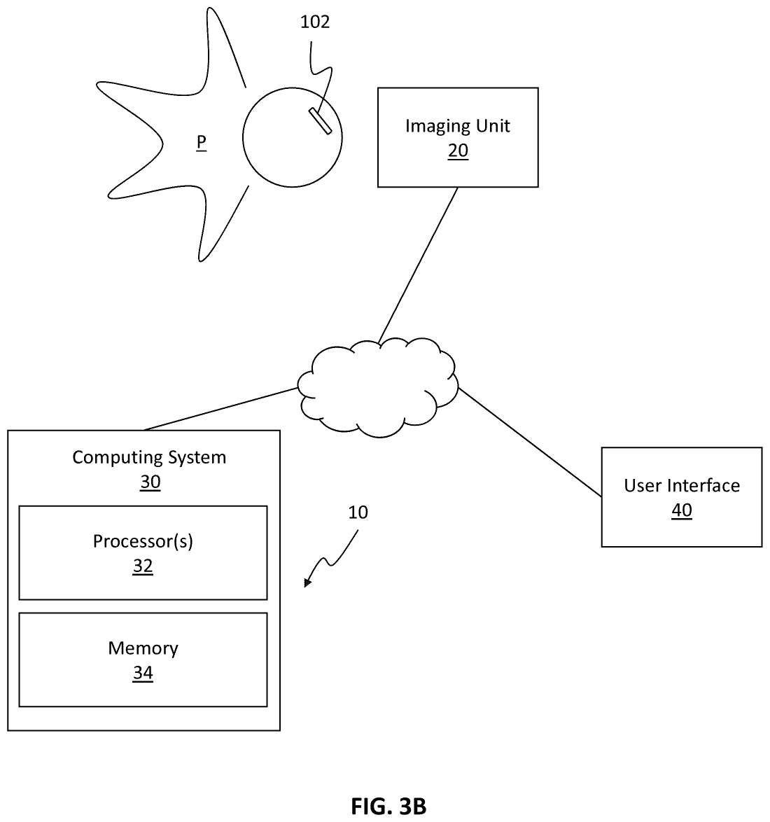

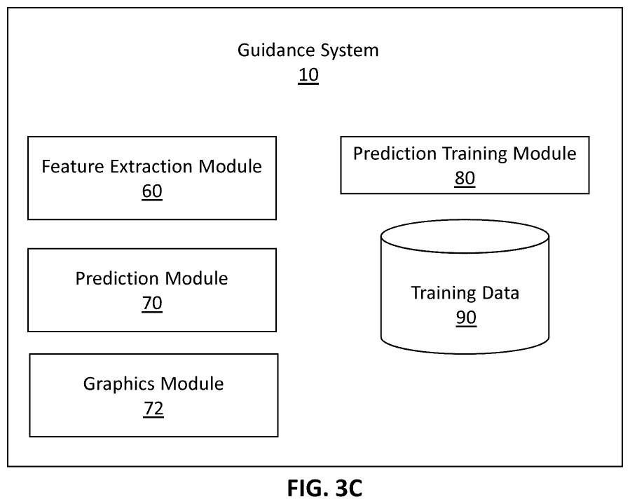

As described above, the thrombectomy device 102 can have a collapsed configuration and an expanded configuration. For example, the thrombectomy device 102 can have a first diameter in the collapsed configuration (e.g., as shown in FIG. 3), and a second diameter in the expanded configuration (e.g., as shown in FIG. 1A), where the second diameter is larger than the first diameter. The thrombectomy device 102 in the collapsed configuration can, for example, be suitable for delivery within the catheter 107 to the target treatment site in a vessel. The thrombectomy device 102 in the expanded configuration can, for example, be suitable for expanding into apposition against the vessel wall, such as for capturing or otherwise engaging a thrombus. The thrombectomy device 102 can be self-expanding, (e.g., by super-elasticity or shape memory), and/or expandable in response to forces applied on the expandable member (e.g., by a balloon). In some variations, the thrombectomy device 102 can be oversized relative to the interior of a vessel in which it is to be used (e.g., the thrombectomy device 102 can be configured to have a larger diameter when allowed to expand outside a vessel, compared to when allowed to expand inside a vessel). In other words, in some variations the thrombectomy device 102 may be sized such that the vessel limits the diameter of some or all of the thrombectomy device 102 placed in the vessel.

As shown in FIG. 1A, the thrombectomy device 102 can have a tubular shape in the expanded configuration, such as with a circular or elliptical cross-section, or other suitable cross-sectional shape. However, in some variations, the thrombectomy device can have a shape that is neither tubular nor cylindrical. In some variations, the thrombectomy device 102 can have a closed proximal end and an open distal end, such as that shown in FIG. 1A. However, in some variations the thrombectomy device 102 can have closed proximal and distal ends, or an open proximal end and a closed distal end. In some variations, at least a portion of the thrombectomy device 102 can include a repeating pattern of structural features. For example, as shown in FIG. 1A, the thrombectomy device can include a matrix of cells. However, in some variations the repeating pattern of structural features can have other forms.

Upon expansion of the thrombectomy device 102 into the expanded configuration, one or more portions of the thrombectomy device can engage a thrombus (e.g., penetrate, interlock with, and/or otherwise capture the thrombus, etc.). In some variations, the thrombectomy device 102 can engage the thrombus with an exterior feature or surface of the expanded thrombectomy device 102. Additionally or alternatively, in some variations, the thrombectomy device 102 can the thrombus with an interior feature or surface of the expanded thrombectomy device 102.

In some variations, the thrombectomy device 102 can be curled, rolled, or otherwise formed from a substantially flat structure. For example, FIG. 1B illustrates an example variation of a vascular device or thrombectomy device 102 including a frame 108 in a flat configuration. In some variations, the frame 108 can include a first edge 124 and a second edge 126 that are opposite one another. The first edge 124 and second edge 126 can be formed, for example, by cutting a sheet or a tube. While the first and second edges are shown as having an undulating, or sinuous configuration, in some variations the first and second edges can have a straight, or linear configuration, or other configuration.

The frame 108 can further include a plurality of struts 114 and a plurality of cells 116 forming a mesh structure. Groups of longitudinally and serially interconnected struts 114 can form undulating members that extend in a generally longitudinal direction. The struts 114 can be connected to each other by joints 120. While the struts are shown having a particular undulating or sinusoidal configurations, in some embodiments the struts can have other configurations. Like the first and second edges 124, 126, the struts 114 and/or cells 116 can be formed, for example, by cutting a sheet or a tube.

The first edge 124 and a second edge 126 may be brought towards one another such that they touch one another, overlap one another, or form a gap between each other (e.g., when the thrombectomy device 102 is in a radially collapsed configuration). For example, when the thrombectomy device 102 is in the radially collapsed configuration, portions of the frame 108 can overlap to facilitate introduction of the thrombectomy device 102 into and through the catheter 107. The extent of any overlap of the frame 108 when the thrombectomy device 102 is in the radially expanded configuration can depend upon a degree of the frame's expansion. Expansion within a vessel can be limited, at least in part, by the vessel's size, and the amount and the properties of any thrombus present. For example, a greater overlap of the edges 124, 126 can occur in narrower vessels, whereas in wider vessels the overlap can be smaller, or even an “underlap” may occur, in which case the edges 124 and 126 are separated by an open gap or space within the vessel. Advantageously, the presence of such an overlap or “roll-up” configuration for the thrombectomy device 102 allows the thrombectomy device 102 to be expanded or compressed in diameter with little or no change in length (e.g., little to no foreshortening during expansion), in comparison to a device that lacks the overlap or roll-up configuration. This is because the expansion or compression can result from a decrease or increase in degree of overlap rather than wholly from deformation of the struts 114 and cells 116, where such deformation can decrease or increase the length of the device when transitioning between the collapsed and expanded configurations.

As shown in FIG. 1A, the thrombectomy device 102 can include a working length 144 and a non-working length 144. The portion of the thrombectomy device 102 in the working length can include a first portion of the cells 116, and is configured to engage a thrombus. The portion of the thrombectomy device in the non-working length 145 can include a second portion of the cells 116, and may also at least contact thrombotic material when the thrombectomy device 102 is in use, but may be less effective than the working length 144 for engaging a thrombus for thrombus capture. In some variations, the non-working length 145 is disposed between the working length and the connection 106 to the manipulation member 104.

In some variations, the non-working length 145 can include a tapered proximal portion 122 (as shown in FIG. 1B) of the thrombectomy device 102, where the proximal portion 122 of the thrombectomy device 102 can be tapered toward a proximal end 110 of the thrombectomy device 102. In some variations, the taper of the proximal, non-working portion 122 can advantageously facilitate retraction and repositioning of the medical device 100 and thrombectomy device 102. For example, in some variations, the non-working length 145 facilitates a retraction of the thrombectomy device 102 into the catheter 107. In some variations, the tapered proximal, non-working portion 122 can be additionally or alternatively designed to generally not contact the vessel wall during a blood flow restoration procedure, and to generally not interfere with the flow of blood within a vessel. The taper of proximal portion 122 can be at various angles relative to the manipulation member 104 or the longitudinal axis of the thrombectomy device 102. For example, in some variations, the taper can have an angle of approximately 45 degrees relative to the manipulation member, though other angles are also possible, and within the scope of the present disclosure. In some variations, the edges 124, 126 of the frame 108 can be curved, straight, or a combination thereof along the tapered proximal portion 122.

As shown in FIGS. 1A and 1B, the thrombectomy device 102 can include multiple marker elements 150 (also referred to herein as “markers”) arranged on the frame 108. In some variations, the marker 150 can include an interior channel, an interior recess or another mounting feature for coupling to the frame 108. For example, the marker 150 can include a band or other substantially tubular element having an open or closed circumference, a coil, or another form configured to couple to the frame 108. For example, in some variations, the marker 150 can be mounted on a strut 114, a joint 120, a feature attached to a strut 114, a feature attached to a joint 120, or any combination thereof. For example, the marker 150 can be mounted on a marker-mounting projection that is attached to a strut. As another example, the marker 150 can be attached to a distally extending tip 154 that can extend from one or more struts 114 and/or joints 120 (described in further detail below).

In some variations, the marker 150 can include a radiopaque material such as platinum, iridium, tantalum, gold, alloys thereof or bismuth and tungsten-doped polymers, among other materials. The marker 150 can be more radiopaque than a body of the vascular or thrombectomy device 102. The marker 150 can be visible under fluoroscopy, CT, X-Rays, MRI, ultrasound technology or other types of imaging.

The markers 150 can be arranged so as to be distributed circumferentially around the thrombectomy device 102 and/or distributed longitudinally along the length of the formed thrombectomy device 102 (e.g., in the roll-up configuration). The markers 150 can be equally or unequally distributed in the circumferential and/or longitudinal directions.

In some variations, the one or more markers 150 can be located at a proximal end 146 of the working length 144, at a distal end 147 of the working length 144, and/or at an intermediate region 149 of the working length 144 between the proximal end 146 and the distal end 147. The working length 144 can extend continuously or intermittently between the proximal end 146 and the distal end 147.

In some variations, the proximal end 146 of the working length 144 can be at a proximalmost location where the thrombectomy device forms a complete circumference. In some variations, the proximal end 146 of the working length 144 can be at a proximalmost location of a region where the thrombectomy device 102 has its greatest transverse dimension in a fully expanded configuration. In some variations, the proximal end 146 of the working length 144 can be at a proximalmost location where the thrombectomy device 102 has a peak, crown, or crest when in a fully expanded state.

In some variations, the distal end 147 of the working length 144 can be at a distalmost location where the thrombectomy device forms a complete circumference. In some variations, the distal end 147 of the working length 144 can be at a distalmost location of a region where the thrombectomy device 102 has its greatest transverse dimension in a fully expanded state. In some variations, the distal end 147 of the working length 144 can be at a distalmost location where the thrombectomy device 102 has a peak, crown, or crest when in a fully expanded state. Furthermore, in some variations, the distal end 147 of the working length 144 can include one or more distally extending tips extending from a distal end of the thrombectomy device 102. For example, the device illustrated in FIG. 1B is shown including four elongate, distally extending tips 154 extending from a distal end of the thrombectomy device 102. In some variations in which the thrombectomy device includes struts, these distal tips 154 can extend from a distalmost row of struts, for example as illustrated in FIG. 1B.

In some variations, a marker 150 located at the proximal end 146 of the working length 144 can be located within 5 mm, within 4 mm, within 3 mm, within 2 mm, or within 1 mm, proximally or distally, of the proximal end 146 of the working length 144. In some variations, a marker 150 located at the proximal end 146 of the working length 144 can be located within the length of one cell or one strut, proximally or distally, of the proximal end 146.

Additionally or alternatively, in some variations, a marker 150 located at the distal end 147 of the working length 144 can be located within 5 mm, within 4 mm, within 3 mm, within 2 mm, or within 1 mm, proximally or distally, of the distal end 147 of the working length 144. In some variations, a marker 150 located at the distal end 147 of the working length 144 can be located within the length of one cell or one strut, proximally or distally, of the distal end 147. Additionally or alternatively, in some variations, the vascular or thrombectomy device 102 can comprise one or more markers 150 attached to the distal tips 154, if present. In some variations in which one or more markers 150 are attached to the distal tips 154, the marker(s) 150 on the distal tips 154 can be positioned at the distal end 147 of the working length 144, for example as illustrated in FIG. 1B.

Additionally or alternatively, in some variations, one or more markers 150 can be located at one or more axial positions along the body of the intermediate region 149 of the working length 144. In some variations, markers 150 may be arranged at one, two, three, or more axial positions along the intermediate region 149. For example, a first group of marker(s) 140 can be located at a first axial position between the proximal end 146 and the distal end 147 of the working length, and a second group of marker(s) 140 can be located at a second axial position between the proximal end 146 and the distal end 147 of the working length, where the first and second axial positions are spaced apart from one another.

A group of markers 150 can be located at some or all of the proximal end 146, the distal end 147, or the intermediate region 149 of the working length 144. In some variations, the group of markers 150 at each of these locations (if present) has a common pattern. For example, a first group of markers 150 at the proximal end 146 can have the same circumferential arrangement relative to each other as a second group of markers 150 at the distal end 147. Additionally or alternatively, some or all of one or more groups of markers 150 at the intermediate region 149 (if present) can have the same circumferential arrangement relative to each other as a group of markers 150 at the proximal end 146 and/or a group of markers 150 at the distal end 147, for example as illustrated in FIG. 1B. However, in some variations, the arrangement of any two distinct groups of markers 150 at different axial locations of the working length 144 can be different. For example, group(s) of markers 150 at the proximal end 146 and at the intermediate region 149 of the working length may have a different circumferential arrangement than a group of markers 150 at the distal end 147 of the working length.

In some variations, the markers 150 can be arranged farther from each other when the thrombectomy device 102 is in an expanded configuration compared to when the thrombectomy device 102 is in collapsed configuration.

In some variations, in addition to or alternatively to having one or more markers 150, some or all of the frame 108 of the thrombectomy device 102 can include a radiopaque material such as platinum, iridium, tantalum, gold, alloys thereof or bismuth and tungsten-doped polymers, among other materials. For example, at least a portion of the struts 114 and/or at least a portion of the joints 120 may include a radiopaque material. At least the radiopaque portion of the frame 108 can be visible under fluoroscopy, CT, X-Rays, MRI, ultrasound technology or other types of imaging. Accordingly, in some variations at least a portion of the frame 108 and/or one or more markers 150 can be radiopaque.

Although the thrombectomy device 102 is primarily described above as having a roll-up configuration, in some variations, the thrombectomy device 102 is circumferentially continuous (e.g., forming a circumferentially continuous tubular or cylindrical shape), lacking first and second edges 124, 126 and having no overlap or gap in a volume-reduced form and expanded form. Regardless of whether the thrombectomy device is circumferentially continuous, the thrombectomy device 102 can have a central longitudinal axis both while in a radially collapsed form and when fully or partially expanded. In some variations, the thrombectomy device 102 can be self-expandable, and can expand toward an expanded configuration upon release from the catheter 107. Upon expansion, the thrombectomy device 102 can expand towards an inner wall of a vessel, towards an occlusive or partially-occlusive thrombus, clot or embolus within a vessel, or both.

Additionally or alternatively, in some variations, the thrombectomy device 102 can include a series of multiple structures. For example, the thrombectomy device 102 can include a series of longitudinal structures, each having proximal and distal ends that are open or closed. Such a series of longitudinal structures can have overlapping longitudinal edges, can be directly coupled to one another (e.g., welded or bonded), and/or secured to one another with one or more additional elements (e.g., a tubular or cylindrical structure arranged within or around the longitudinal structures). As another example, the thrombectomy device 102 can include a series of tubular structures that have overlapping circumferential edges, can be directly coupled to one another (e.g., welded or bonded), and/or secured to one another with one or more additional elements (e.g., a longitudinal structure arranged within or around the tubular structures).

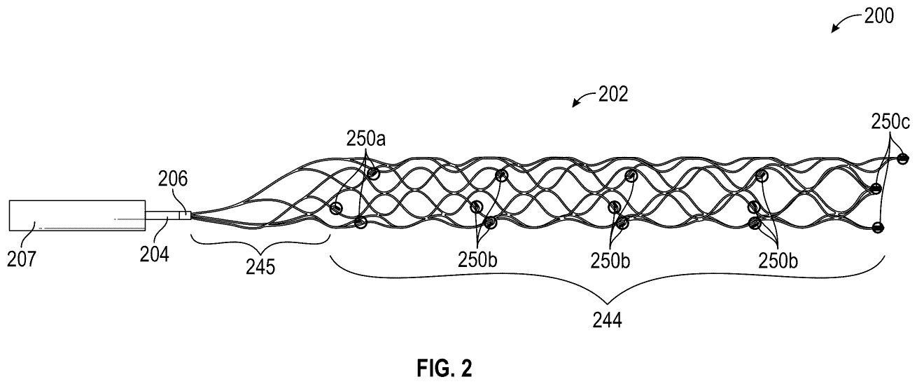

FIG. 2 illustrates an example variation of a medical device 200 (e.g., thrombectomy system) including a vessel device or thrombectomy device 202 that is similar to the thrombectomy system 100 and thrombectomy device 102, respectively, except as described below. For example, like the thrombectomy system 100, the thrombectomy device 200 can include a manipulation member 204 and a thrombectomy device 202, which can be placed in a catheter 207 such as for delivery to a target treatment site. The thrombectomy device 202 can be attached to the manipulation member 204 with a connection 206 similar to connection 106. The connection 206 can include a radiopaque marker.

Like the thrombectomy device 102, the thrombectomy device 202 can include a working length 244 and a non-working length 245, with a plurality of markers 250 (e.g., collectively 250a, 250b, and 250c). As shown in FIG. 2, the thrombectomy device 202 can include a group of markers 250a located at a proximal end of the working length 244, three groups of markers 250b located at various axial locations in an intermediate region of the working length 244, and a group of markers 250c located at a distal end of the working length 244 (at distal tips that extend distally from the distalmost row of struts and/or joints in the thrombectomy device 202). In an example variation, two adjacent groups of markers (e.g., markers 250a and a proximalmost group of markers 250b, or two adjacent groups of markers 250b) can be longitudinally separated from each other by an axial distance of between about 3 mm and about 12 mm, or between about 5 mm and about 10 mm, or about 5 mm, or about 7 mm, or about 10 mm. However, the groups of markers can be separated by any suitable distance. Each of these groups of markers 250, as shown, includes three circumferentially-distributed markers. However, it should be understood that in other variations, the thrombectomy device 202 can include any suitable number of groups of markers 250 (e.g., two, three, four, five, or more), and each group of markers 250 can include any suitable number of markers (e.g., two, three, four, five or more, etc.).

### II. Thrombectomy Guidance

FIG. 3A is a schematic flowchart of a method 400 for guiding blood flow restoration in a vessel, such as removal (e.g., retrieval) of a thrombus. The method 400 is primarily described herein in connection with guiding a thrombectomy procedure. For example, in some variations, the method 400 can be used to guide a thrombectomy performed with any of the thrombectomy devices described herein (e.g., medical device 100, medical device 200). However, the method 400 can be used to guide a thrombectomy performed with any suitable vessel device. Furthermore, it should be understood that other variations of the method can be performed with respect to other suitable blood flow restoration procedures involving the vessel device or a thrombectomy device.

As shown in FIG. 3A, the method 300 can include receiving image data indicative of relative positions of marker elements on a thrombectomy device positioned proximate a thrombus in a patient 300, and predicting at least one characteristic of a thrombus 320 at least in part by applying a trained machine learning algorithm to the image data. In some variations, the method 310 can further include providing the predicted characteristic of the thrombus to a user 330 (e.g., clinician performing a thrombectomy procedure with the thrombectomy device). The predicted characteristic of the thrombus can then be used to prompt one or more prophylactic actions to reduce the chance of a non-optimum patient outcome. For example, based at least in part on the predicted characteristic of the thrombus, a user (e.g., clinician) may modify the thrombectomy procedure as appropriate, to improve patient outcome.

The method 300 is primarily described herein with respect to analyzing relative positions of marker elements on a thrombectomy device visible in image data. However, as described above, in some variations, other portions of the thrombectomy device may be visible in imaging (e.g., some or all of frame 108). As such, it should be understood that in some variations, the method may additionally or alternatively include receiving image data indicative of a shape of a thrombectomy device (e.g., shape of a frame of the thrombectomy device) and predicting at least one characteristic of a thrombus at least in part by applying a trained machine learning algorithm to such image data.

Advantageously, the method 300 allows for assessment or other characterization of a thrombus using image(s) of the thrombectomy device, without directly visualizing the thrombus itself. For example, the images can include fluoroscopic images. While fluoroscopy can be used to help position and track the thrombectomy device, the thrombus itself cannot be visualized under fluoroscopy. Accordingly, the method 300 enables creation of a digital representation of the thrombus using image data that is readily available during a thrombectomy or other blood flow restoration procedure. One or more features of the image data depicting markers on a thrombectomy device (and/or other portions of the thrombectomy device) can be transformed into such a digital representation of the thrombus (e.g., incorporating one or more predicted characteristics of the thrombus), even though the thrombus itself may otherwise not be visualized in the original image data.

The digital representation of the thrombus can help guide one or more aspects of the procedure. For example, based on the digital representation of the thrombus (e.g., a predicted characteristic of the thrombus), a user may proceed with the procedure as currently planned, may reposition the thrombectomy device, may collapse and re-expand the thrombectomy device, may adjust the trajectory of the withdrawal of the thrombectomy device when the thrombectomy device is at least partially engaged with the thrombus, and/or the like. In some variations, the method 300 helps increase the first pass efficacy of the thrombectomy device, such as by reducing the likelihood of a needing to perform a second attempt for thrombus removal after the thrombectomy device is withdrawn in the first attempt.

Accordingly, in some variations the method 300 can be performed intra-operatively and/or substantially in real-time in order to predict a characteristic of a thrombus during a blood flow restoration procedure (e.g., thrombectomy). For example, at least a portion of the method 300 may be performed by a guidance system 10 as shown in the schematic illustration of FIG. 3B. As shown in FIG. 3B, a guidance system 10 for a vessel device or thrombectomy device 102 can include a computing system 30 including at least one processor 32 and a memory 34 that is operably coupled to the at least one processor 32, where the memory 34 stores instructions that, when executed by the at least one processor 32, cause the computing system to perform operations to predict at least one characteristic of a thrombus (e.g., with a machine learning algorithm configured to analyze image data of the thrombectomy device 102). At least a portion of the computing system 30 may, for example, be located in an operating room during a blood flow restoration procedure, and/or at least a portion of the computing system 30 may be located remote from the operating room.

Furthermore, the guidance system 10 can be operably connected to an imaging unit 20 and/or a user interface 40. The guidance system 10, imaging unit 20, and/or user interface 40 can be operably connected with each other, such as over a wired or wireless network. Alternatively, in some variations at least a portion of the guidance system 10 can be embodied in the imaging unit 20 itself. For example, the imaging unit 20 can include at least one processor 32 and/or memory 34 where the memory 34 stores instructions that, when executed by the at least one processor 32, cause the computing system to perform operations to predict at least one characteristic of a thrombus (e.g., with a machine learning algorithm configured to analyze image data of the thrombectomy device 102). In some variations, the imaging unit 20 can be a fluoroscopic imaging unit configured to generate fluoroscopy images (e.g., angiography system). The images can be static images and/or part of a dynamic video feed. However, the imaging unit 20 can additionally or alternatively include one or more other suitable imaging modalities, such as CT, MRI, ultrasound, etc. The user interface 40 can be configured to provide to a user information relating to the blood flow restoration procedure. For example, in some variations, the user interface 40 can include a display configured to such as images generated by the imaging unit 20 and/or predicted characteristic(s) of the thrombus (e.g., as predicted by the guidance system 10 in accordance with the methods described herein), such as a predicted boundary of the thrombus). Furthermore, in some variations the user interface 40 can receive one or more commands or other inputs from the user. For example, the user interface 40 can be configured to enable a user to annotate an image or digital representation of the thrombus, control the imaging unit 20, and/or the like.

FIG. 3C is a schematic illustration including further details of an example variation of a guidance system 10 for a blood flow restoration procedure. The guidance system 10 can include a prediction module 70 configured to predict one or more characteristics of a thrombus based at least in part on images inputted into a trained prediction machine learning algorithm. In some variations, the guidance system 10 can include a feature extraction module 60 configured to extract features from received images of a thrombectomy device, for use by the prediction module 07 and/or the prediction training module 80. However, the guidance system 10 can omit the feature extraction module 60 (e.g., if a machine learning algorithm utilized by the prediction module 70 or prediction training module 80 does not require extracted features as input). Furthermore, in some variations, the guidance system can include a prediction training module 80 and storage of training data 90, which can be configured to train the machine learning algorithm utilized by the prediction module 70. However, in some variations the guidance system can omit the prediction training module 80 and storage of training data 90. One or more of the feature extraction module 60, prediction module, prediction training module 80, and/or storage of training data 90 can be embodied in the computing system 30.

The guidance system 10 can be utilized during a training phase and/or a prediction phase. For example, in the training phase, the feature extraction module 60, the storage of training data 90, and/or the prediction training module 80 can be used to build and train a machine learning algorithm of the prediction module 70. The training data can, for example, include training images depicting the relative positions of markers (and/or other thrombectomy device features visible in the training images) on a thrombectomy device placed in various known scenarios in a vessel. Different training images can, for example, depict markers of a thrombectomy device in various thrombectomy scenarios including but not limited to: full engagement with a thrombus, partial engagement with a thrombus, withdrawal from a vessel while remaining fully engaged with a thrombus, and/or withdrawal from a vessel while experiencing diminishing engagement with a thrombus (e.g., due to pieces of the thrombus breaking off, etc.). Different training images can depict markers and/or shape of a thrombectomy device in such varied scenarios occurring in different vessels having various sizes and/or shapes with respect to thrombi having various sizes and/or shapes. Additionally or alternatively, different training images can depict markers and/or shape of a thrombectomy device in one or more various vessels while contacting, engaging, and/or otherwise interacting with various thrombi having various material characteristics, such as stiffness (which may vary among different thrombi at least in part on amount of fibrinogen or calcification, for example). In some variations, a material characteristic such as stiffness can be quantified for thrombi in the training images, such as on a numerical scale or graded on a letter scale.

The training data can be applied by the prediction training module 80 to train a suitable machine learning algorithm to analyze the relative positions of the markers and/or contours of the thrombectomy device for the various thrombectomy scenarios. For example, the feature extraction module 60 can be configured to extract features from the training images and provide the extracted features to the prediction training module 80 for training one or more machine learning algorithms for the prediction module 70. The feature extraction module 60 can, for example, be configured to extract the locations (and relative positioning) of the markers and/or contours of the imaged thrombectomy device as depicted in the training images. The feature extraction module can, for example, include one or more suitable computer vision algorithms. The extracted features can be used by the prediction training module 80 to train a prediction machine learning algorithm that analyzes the extracted features to predict a characteristic of the thrombus (e.g., boundary of the thrombus, material characteristic of the thrombus, etc.).

The trained machine learning algorithm include one or more suitable software algorithms, such as rule-based algorithms, machine learning algorithms, or combinations thereof. Examples of machine learning algorithms that may be used include: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning), regularization algorithms (e.g., ridge regression, least absolute shrinkage and selection operator, clastic net, least-angle regression), decision tree algorithms (e.g., Iterative Dichotomiser 3 (ID3), C4.5, C5.0, classification and regression trees, chi-squared automatic interaction detection, decision stump, M5), Bayesian algorithms (e.g., naïve Bayes, Gaussian naïve Bayes, multinomial naïve Bayes, averaged one-dependence estimators, Bayesian belief networks, Bayesian networks, hidden Markov models, conditional random fields), clustering algorithms (e.g., k-means, single-linkage clustering, k-medians, expectation maximization, hierarchical clustering, fuzzy clustering, density-based spatial clustering of applications with noise (DBSCAN), ordering points to identify cluster structure (OPTICS), non negative matrix factorization (NMF), latent Dirichlet allocation (LDA), Gaussian mixture model (GMM)), association rule learning algorithms (e.g., apriori algorithm, equivalent class transformation (Eclat) algorithm, frequent pattern (FP) growth), artificial neural network algorithms (e.g., perceptrons, neural networks, back-propagation, Hopfield networks, autoencoders, Boltzmann machines, restricted Boltzmann machines, spiking neural nets, radial basis function networks), deep learning algorithms (e.g., deep Boltzmann machines, deep belief networks, convolutional neural networks, stacked auto-encoders), dimensionality reduction algorithms (e.g., principle component analysis (PCA), independent component analysis (ICA), principle component regression (PCR), partial least squares regression (PLSR), Sammon mapping, multidimensional scaling, projection pursuit, linear discriminant analysis, mixture discriminant analysis, quadratic discriminant analysis, flexible discriminant analysis), ensemble algorithms (e.g., boosting, bootstrapped aggregation, AdaBoost, blending, gradient boosting machines, gradient boosted regression trees, random forest), or suitable combinations thereof. The machine learning algorithms described herein can be trained using any suitable technique, including supervised learning, unsupervised learning, semi-supervised learning, reinforcement learning, or suitable combinations thereof.

The trained machine learning algorithm can be configured to output at least one characteristic of the thrombus based at least in part on the image data. In some variations, the machine learning algorithm can be configured to analyze positions of one or more markers on the thrombectomy device. In some variations, the three-dimensional positions (e.g., the position of one or more markers as defined in a three-dimensional coordinate system) of markers may be analyzed. Additionally or alternatively, in some variations the two-dimensional positions (e.g., the position of one or more markers as defined in a two-dimensional coordinate system) in any of the above-described timepoints, as some characterizations of the thrombus may be predicted based on marker positions as projected onto a two-dimensional plane.

For example, in some variations, the machine learning algorithm can be configured (e.g., trained) to analyze the relative positions of multiple markers at a given point in time (e.g., within a single image). For example, the machine learning algorithm can be configured to predict a thrombus characteristic based on the relative positions of markers when the thrombectomy device is contacting the thrombus, presumed to have engaged with (e.g., captured) the thrombus, or is in the process of being withdrawn from the vessel. In these variations, the machine learning algorithm can, for example, be trained to predict a boundary of the thrombus and/or a material characteristic (e.g., stiffness) of the thrombus based on the relative positions of markers as the thrombectomy device interacts with the thrombus at a single timepoint.

As another example, the machine learning algorithm can be configured (e.g., trained) to analyze a change in the relative positions of multiple markers across two or more timepoints (e.g., within two or more sequential images). For example, the machine learning algorithm can be configured to predict a thrombus characteristic based on how the relative positions of multiple markers change between a first timepoint and a second timepoint. As another example, the machine learning algorithm can be configured to predict a thrombus characteristic based on a trend of how the relative positions of multiple markers change over three or more timepoints. For example, one timepoint can be prior to the thrombectomy device contacting a thrombus, and another timepoint can be after the thrombectomy device contacts the thrombus. As another example, one timepoint can be when the thrombectomy device is expanded a first amount, and another timepoint can be when the thrombectomy device is expanded a second amount greater than the first amount. As yet another example, one timepoint can be when the thrombectomy device (e.g., while engaging the thrombus) is at a first position within vasculature, and another timepoint can be when the thrombectomy device is at a second position within vasculature different from the first position (e.g., different axial locations of a vessel, different rotational orientations within a vessel). In these variations, the machine learning algorithm can, for example, be trained to predict a boundary of the thrombus and/or a material characteristic (e.g., stiffness) of the thrombus based on the change in relative positions of markers as the thrombectomy device interacts with the thrombus across two timepoints, across three timepoints, or more than three timepoints.

In some variations, the machine learning algorithm can additionally or alternatively be configured to analyze the position of a single marker at a given point in time (e.g., within a single image). This can be performed for each of multiple markers. Additionally or alternatively, in some variations, the machine learning algorithm can be configured to analyze a change in position of a single marker across two or more timepoints (e.g., within two or more sequential images). This can be performed for each of multiple markers. For example, the machine learning algorithm can be configured to predict a thrombus characteristic based on how the position of a single marker change between a first timepoint and a second timepoint. As another example, the machine learning algorithm can be configured to predict a thrombus characteristic based on a trend of how the position of a single marker changes over three or more timepoints. For example, one timepoint can be prior to the thrombectomy device contacting a thrombus, and another timepoint can be after the thrombectomy device contacts the thrombus. As another example, one timepoint can be when the thrombectomy device is expanded a first amount, and another timepoint can be when the thrombectomy device is expanded a second amount greater than the first amount. As yet another example, one timepoint can be when the thrombectomy device (e.g., while engaging the thrombus) is at a first position within vasculature, and another timepoint can be when the thrombectomy device is at a second position within vasculature different from the first position (e.g., different axial locations of a vessel, different rotational orientations within a vessel). In these variations, the machine learning algorithm can, for example, be trained to predict a boundary of the thrombus and/or a material characteristic (e.g., stiffness) of the thrombus based on the position of a single marker at any given timepoint, and/or the change in position of a single marker as the thrombectomy device interacts with the thrombus across two timepoints, across three timepoints, or more than three timepoints.

In some variations, the machine learning algorithm can additionally or alternatively be configured to analyze the shape of the thrombectomy device. For example, the algorithm can be configured to analyze one or more contours of the thrombectomy device at a given point in time, and/or a change in one or more contours of the thrombectomy device across two or more timepoints. In some variations, the machine learning algorithm can analyze one or more contour features of a segment of the thrombectomy device, or can analyze one or more contour features of the entire length of the thrombectomy device. Furthermore, in some variations, different individual features of a frame (e.g., certain struts, certain joints) of the thrombectomy can additionally or alternatively be extracted and function as marker elements for analysis by the machine learning algorithm, similar to that described above.

Accordingly, various training data (e.g., in the storage of training data 90 such as that described above) may be inputted into the machine learning algorithm by the prediction training module 80, in order to train a machine learning algorithm utilized by the prediction module 70 to predict certain characteristics of the thrombus.

As described above, the guidance system 10 can additionally or alternatively be utilized during a prediction phase (e.g., intra-operatively, or during a thrombectomy or other blood flow restoration procedure). During the prediction phase, patient images indicative of the relative positions of markers on a thrombectomy device can be received and inputted into the trained prediction machine learning algorithm of the prediction module 70. In some variations, similar to that described above with respect to the training phase, the feature extraction module 60 can be configured to extract features from the received patient images, such as the three-dimensional and/or two-dimensional positions of markers on the thrombectomy device.

The prediction module 70 can be configured to predict one or more characteristics of the thrombus, utilizing the machine learning algorithm trained during the training phase. For example, similar to that described, the prediction module can be configured to predict a characteristic of the thrombus based on any one or more of: (i) relative positions of multiple markers at a given point in time, (ii) change in the relative positions of multiple markers across two or more timepoints, (iii) position of a single marker at a given point in time, and (iv) a change in position of a single marker across two or more timepoints. Additionally or alternatively, the prediction module 70 can be configured to predict a characteristic of the thrombus based on (v) one or more contours (and/or other suitable individual features) of the thrombectomy device at a given point in time, and (vi) one or more contours (and/or other suitable individual features) of the thrombectomy device across two or more timepoints. As discussed elsewhere herein, the prediction module 70 can, in some variations, predict a boundary of a thrombus and/or a material characteristic (e.g., stiffness) of the thrombus.

In some variations, one or more predicted characteristics of the thrombus can be communicated to a user (e.g., clinician performing a thrombectomy procedure with the thrombectomy device), such as in the form of a digital representation of the thrombus. For example, a predicted boundary of the thrombus may be rendered as a graphic (e.g., by a graphics module 72 in the guidance system 10), which may be displayed on the user interface 40 such as on a display screen. In some variations, the graphic of the thrombus boundary can be overlaid over other image data, such as the received image data depicting the markers on the thrombectomy device, and/or other image data. The graphic of the thrombus boundary can, for example, include an outline corresponding to the predicted thrombus boundary (e.g., in solid line, dashed line, colored line, etc.) and/or shading to illustrate thrombus volume within the predicted thrombus boundary. The graphic of the thrombus boundary can be displayed in a two-dimensional manner and/or a three-dimensional manner on the user interface 40. Additionally or alternatively, a predicted material characteristic (e.g., stiffness) of the thrombus can be provided to a user via the user interface 40. For example, a thrombus graphic displayed on the user interface 40 can include shading (e.g., color, patterns) within the predicted thrombus boundary that corresponds to predicted amount of stiffness or other degree of a material characteristic of the thrombus. As another example, a predicted material characteristic of the thrombus can additionally or alternatively be provided to a user as a grade on a numerical, letter, and/or color scale separately from a graphic of the thrombus boundary (e.g., displayed on the user interface 40 and/or communicated as audio output, etc.).

The predicted characteristic of the thrombus can then be used to prompt one or more prophylactic actions to reduce the chance of a non-optimum patient outcome. For example, based at least in part on the predicted characteristic of the thrombus, a user (e.g., clinician) may modify the thrombectomy procedure as appropriate, to improve patient outcome.

### III. Example Guided Thrombectomy

An example thrombectomy procedure in accordance with the present technology is described below. Although the procedure is described with reference to thrombectomy system 100 with thrombectomy device 102, it should be understood that the description similarly applies to operation of the thrombectomy system 200 with thrombectomy device 202, and/or other variations in accordance with the present technology.

Receiving image data indicative of relative positions of marker elements on a thrombectomy device functions to obtain information that can be analyzed by one or more machine learning algorithms to predict at least one characteristic of the thrombus. In some variations, the image data includes one or more fluoroscopic images. However, other imaging modalities (e.g., CT, MRI, ultrasound) may additionally or alternatively be received for analysis, such as for generating supplemental assessment of the thrombus. In some variations, the image data can include two-dimensional image(s), or the image data can additionally or alternatively include three-dimensional image(s). The image data can include one or more static images taken at discrete timepoints (e.g., every second, every two seconds, every five seconds, etc.) and/or at selected phases or milestone steps of the blood flow restoration procedure.

In some variations, image data can be obtained and analyzed throughout a thrombectomy procedure. For example, some image(s) may depict the vessel without the thrombectomy device present, some of image(s) may depict the thrombectomy device prior to expansion or deployment of the thrombectomy device and/or during expansion or deployment of the thrombectomy device, and/or some of the image(s) may depict the thrombectomy device as it is withdrawn from the vessel. For example, in some variations, received image data can depict the vessel and/or a medical device (including, for example, a thrombectomy device) during one or more portions of a thrombectomy described herein.

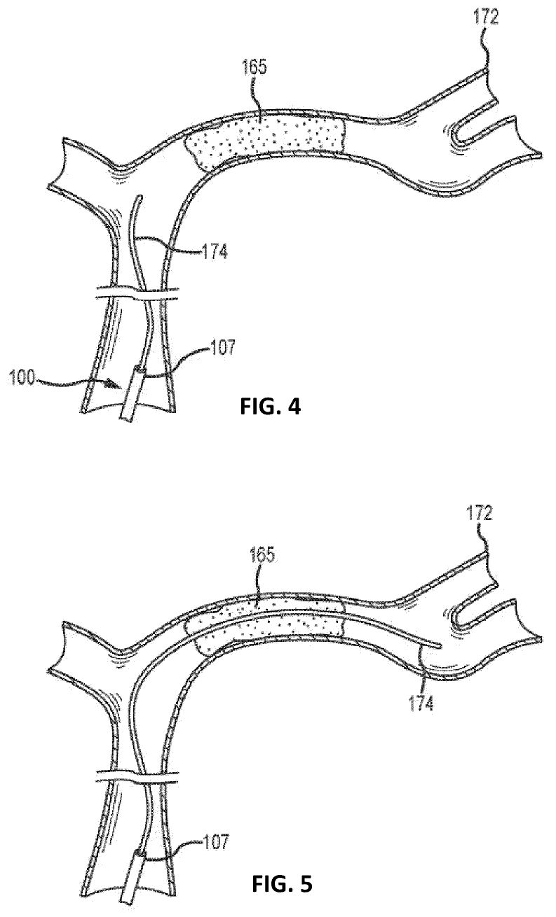

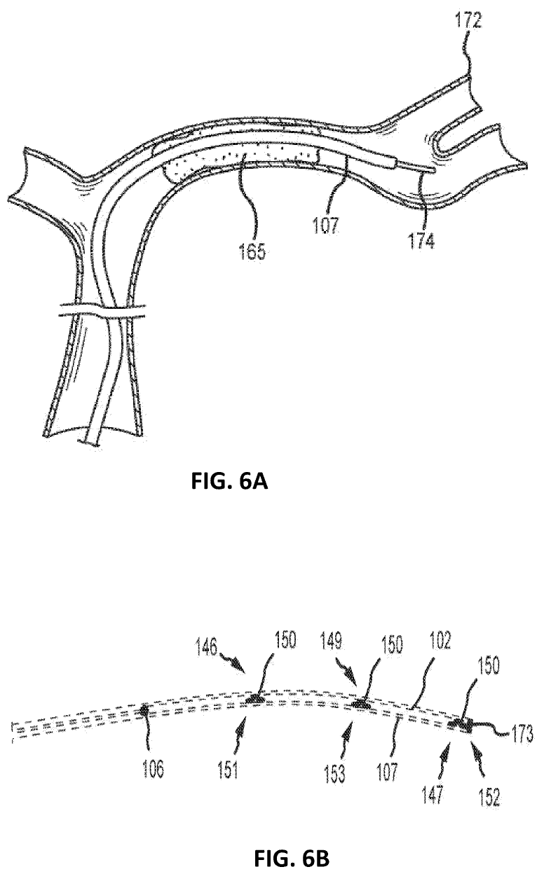

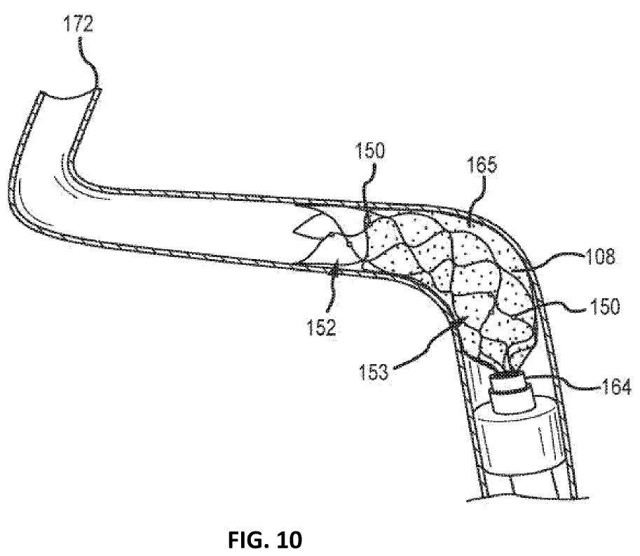

As shown in FIG. 4, a medical device 100 may be inserted into an anatomical vessel 172 by first inserting a guide wire 174 into the anatomical vessel 172. The illustrated anatomical vessel is an intracranial blood vessel, though can be any other suitable vessel for treatment. In some variations, the medical device is introduced into a segment of cerebral blood vessel distal to the carotid siphon. The guide wire 174 can be advanced through a guide catheter 164 (see FIG. 10), which optionally includes a balloon near the guide catheter's distal end, and/or a catheter 107 to the treatment site, until the guide wire 174 is adjacent the thrombus 165. As shown in FIG. 5, the guide wire 174 is further advanced distally through the thrombus 165. Once the guide wire 174 is in position, the catheter 107 is advanced over the guide wire 174, through a distal end of the guide catheter, toward the thrombus 165 in the anatomical vessel 172. As shown in FIG. 6A, the catheter 107 is advanced distally through the thrombus 165. The guide wire 174 is then withdrawn proximally.

The medical device 100 is advanced through the catheter 107 that has been advanced through the thrombus. The medical device 100 is advanced through the catheter 107 by the manipulation member 104 coupled to the thrombectomy device 102 (e.g., at the proximal end of the thrombectomy device). The catheter 107 may substantially prevent expansion of the thrombectomy device 102 and thus maintain the thrombectomy device 102 in a compressed, radially collapsed configuration as the thrombectomy device 102 is advanced to the treatment site within the catheter 107.

FIG. 6B is a schematic representation of a fluoroscopic image of an arrangement of imaged markers when an example variation of a thrombectomy device 102 is in the radially collapsed configuration within the catheter 107. A marker at connection 106 and/or a marker 173 at the distal end of the catheter 107 can be visualized in the fluoroscopic image. Furthermore, the fluoroscopic image can show a first portion of markers 150 in a proximal marker group 151 located at the proximal end 146 of the working length 144, a second portion of markers 150 in a distal marker group 152 located at the distal end 147 of the working length 144, and/or additional markers in one or more intermediate marker groups 153 located in the intermediate region 149 of the working length 144. The markers 150 in each group can be visualized as in close lateral proximity to each other (e.g., generally at the same axial location along the working length 144 of the thrombectomy device 102).

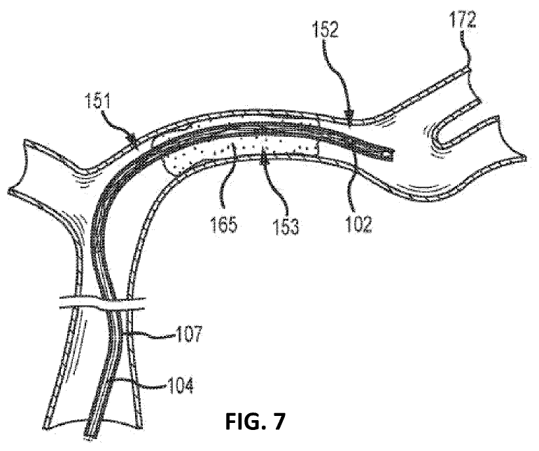

The thrombectomy device 102 can be advanced or otherwise moved to position one or more of the marker groups to a desired position relative to the anticipated position of the thrombus (which may, for example, be previously identified through prior imaging, such as before the thrombectomy procedure). For example, as shown in FIG. 7, in some variations, the thrombectomy device 102 can be placed so as to simultaneously (i) position the proximal marker group 151 proximal to an anticipated proximal end 170 of the thrombus 165, and (ii) position the distal marker group 152 distal to an anticipated distal end 171 of the thrombus 165. If an intermediate marker group 153 is present, the thrombectomy device 102 can be advanced or otherwise moved to position the intermediate marker group 153 between the anticipated proximal and distal ends of (e.g., within) the thrombus.

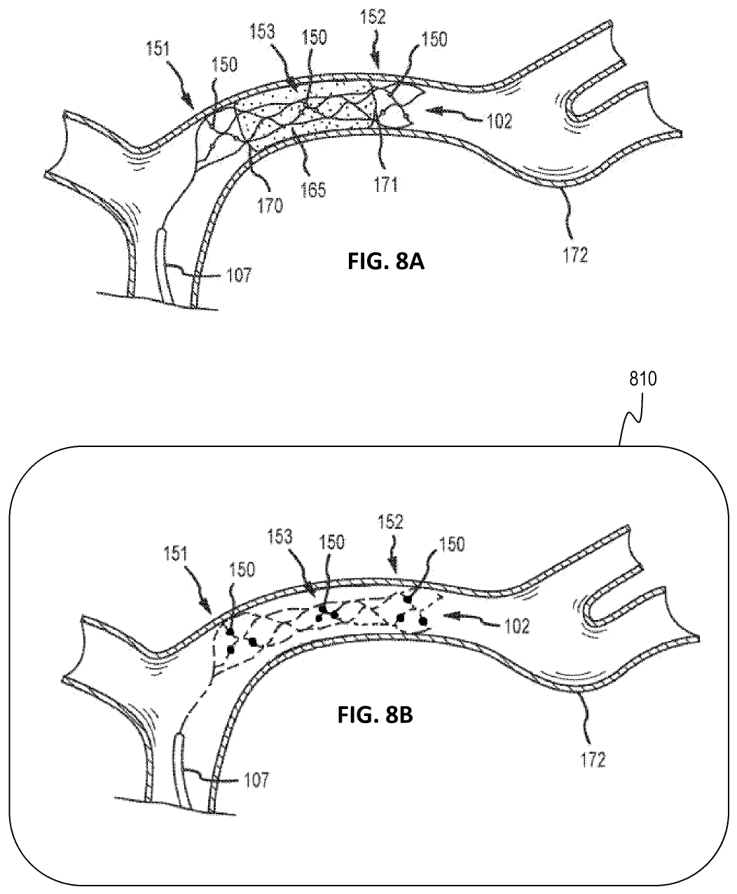

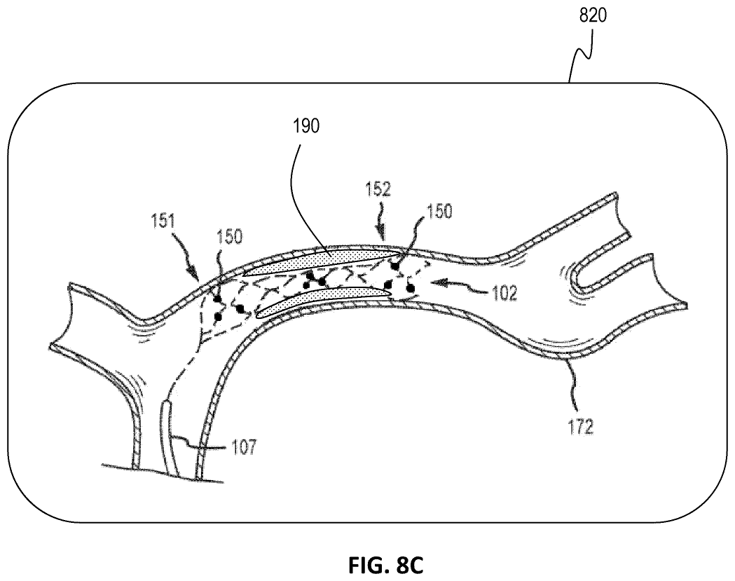

The catheter 107 can then be withdrawn proximally relative to the thrombectomy device 102 to at least partially expose the thrombectomy device 102. If the thrombectomy device 102 is self-expanding, retraction of the catheter 107 can permit the thrombectomy device 102 to expand to a radially expanded state. For example, FIG. 8A illustrates the thrombectomy device 102 fully exposed by proximal retraction of the catheter 107, after being positioned relative to the thrombus 165 at an example location as described above with respect to FIG. 7. As shown in FIG. 8A, at this example device location, the proximal and distal portions of the thrombectomy device 102 are more radially expanded than the intermediate region that engages the thrombus 165. This is because at this example device location, a proximal portion of the thrombectomy device 102 is located proximal to the thrombus 165 (e.g., proximal to a proximal end 170 of the thrombus), and a distal portion of the thrombectomy device 102 is located distal to the thrombus 165 (e.g., distal to a distal end 171 of the thrombus). The proximal and distal portions of the thrombectomy device 102 can undergo a greater radial expansion unimpeded by the thrombus 165, while the presence of the thrombus 165 may impede or inhibit fuller radial expansion of the intermediate region of the thrombectomy device 102. However, because the thrombus 165 itself may not be visualized under imaging (e.g., fluoroscopic imaging), the desired engagement between the thrombectomy device 102 and the thrombus 165 might not necessarily be confirmable by a user directly viewing the imaging.

In accordance with the present technology, receiving image data indicative of relative positions of marker elements on a thrombectomy device 310 can include receiving image data indicative of relative positions of markers 150. For example, as shown in FIG. 8B, at least one image 810 can be obtained, where the image 810 depicts the various marker groups of markers 150 (the outline of the thrombectomy device 102 is depicted in broken line for reference, and may or may not be visible in the image 810 itself). In particular, FIG. 8B shows the markers 150 of the proximal marker group 151 and the distal marker group 152 as more laterally separated than the markers 150 of the intermediate 153 marker group, which appear in a less expanded distribution.

The one or more images 810 can be inputted into a trained machine learning algorithm of the prediction module 70 (and the feature extraction module 60, if present), which can be configured to analyze the images 810 to predict at least one characteristic of the thrombus 165. For example, the feature extraction module 60 can utilize a suitable computer vision algorithm to identify positions of the markers 150 in the images 810, and/or the trained machine learning algorithm of the prediction module 70 can analyze the two-dimensional and/or three-dimensional positions of the markers 150 to predict at least one characteristic of the thrombus. As described elsewhere herein, the prediction module 70 can be configured, for example, to predict a boundary of the thrombus and/or a material characteristic (e.g., stiffness) of the thrombus.

In some variations, providing at least one predicted characteristic of the thrombus to a user 330 can include presenting the at least one predicted characteristic of the thrombus on a user interface device such as a display. For example, as shown in FIG. 8C, the predicted characteristic of the thrombus can include a predicted boundary of the thrombus, which can be rendered as a digital representation 190 of the thrombus by the graphics module 72 to be displayed. In some variations, the predicted boundary of the thrombus can be displayed overlaid with the image(s) under which the thrombectomy procedure is being performed, in a combined or overlay image 820. However, in some variations the predicted boundary of the thrombus can be displayed separately from such imaging. As another example, the predicted characteristic of the thrombus can include a material characteristic (e.g., stiffness) of the thrombus, which can be presented to a user via a display or other suitable user interface. In other examples, the at least one predicted characteristic of the thrombus can additionally or alternatively be provided to the user in any suitable manner, such as any of the manners discussed above.

In some variations, partial retraction of the catheter 107 can permit a portion of the thrombectomy device 102 to expand to a radially expanded state. For example, the catheter 107 can be proximally retracted such that only the distal portion of the thrombectomy device 102 and a portion (e.g., a distal third, a distal half, etc.) of the intermediate region of the thrombectomy device 102 are exposed and allowed to radially expand. In this example, similar to their corresponding portions in the example shown in FIG. 8A, the exposed distal portion of the thrombectomy device 102 may be more radially expanded than the exposed portion of the intermediate region of the thrombectomy device 102 that may be engaged with the thrombus 165. However, similar to that described above with respect to FIG. 8A, the desired engagement between the thrombectomy device 102 and the thrombus 165 might not necessarily be confirmable by a user directly viewing imaging (e.g., fluoroscopic imaging) under which the thrombectomy procedure is being performed. Accordingly, in some variations the method can include receiving image data indicative of relative positions of markers on the exposed portion of the thrombectomy device. Similar to that described above, one or more of such images can be inputted into a trained machine learning algorithm of the prediction module 70 (and the feature extraction module 60, if present), which can be configured to analyze the images 810 to predict at least one characteristic of the thrombus 165 based on the markers on the exposed portion of the thrombectomy device.

A user can use the predicted characteristic(s) of the thrombus to assess whether there is sufficient or otherwise satisfactory engagement between the thrombectomy device 102 and the thrombus 165 (e.g., to provide a high likelihood of success of removing the thrombus from the vessel on the first pass). If not satisfied by the engagement between the thrombectomy device 102, the user can perform corrective or repositioning actions such as adjusting the longitudinal position of the thrombectomy device 102, adjusting the rotational position of the thrombectomy device 102, adjusting the amount of radial expansion (e.g., if the thrombectomy device 102 is expanded via a balloon), and/or the like. In some variations, repositioning or adjusting the thrombectomy device 102 can include re-collapsing the thrombectomy device 102 such as by distally advancing the catheter 107, and re-expanding the thrombectomy device 102 after repositioning the thrombectomy device 102 such as by again proximally retracting the catheter 107 to expose the repositioning thrombectomy device 102.

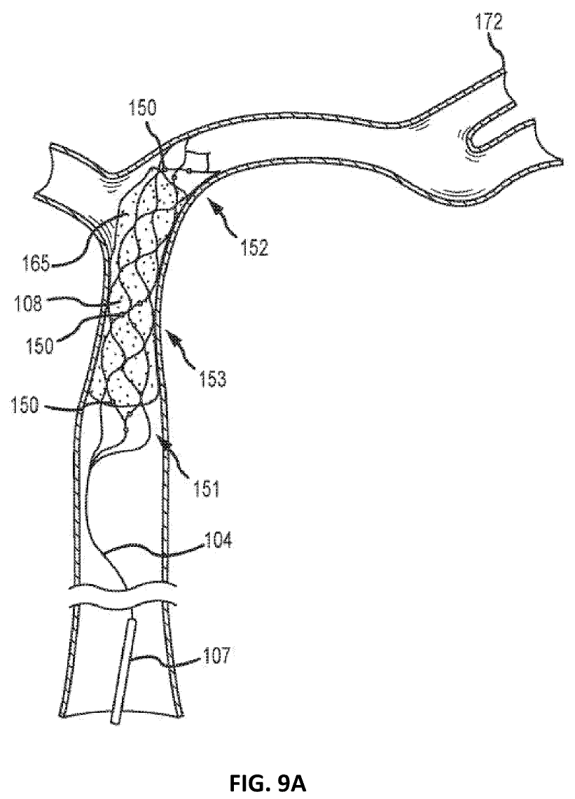

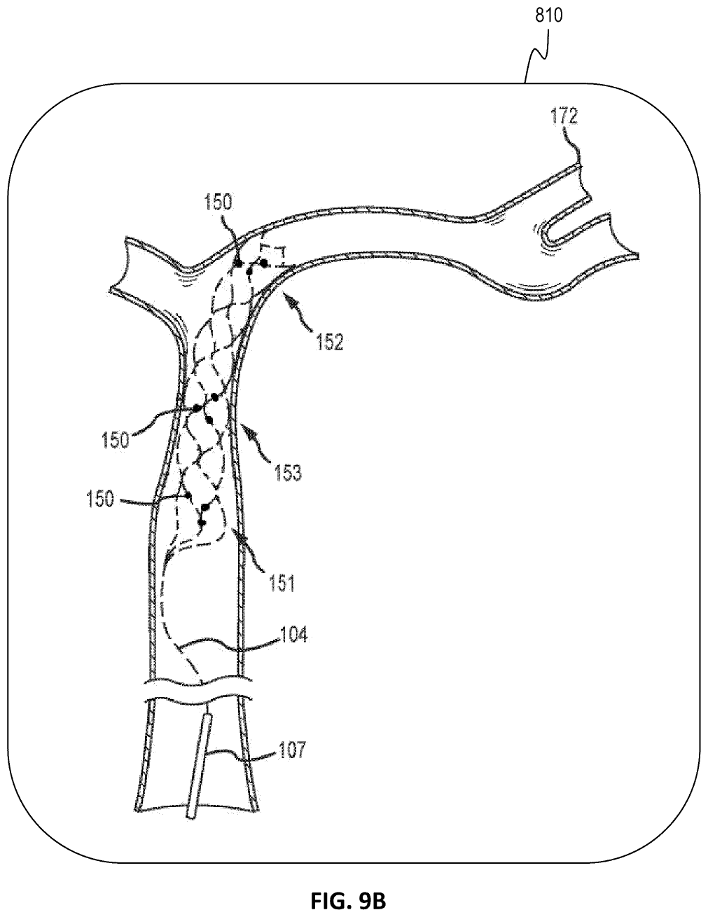

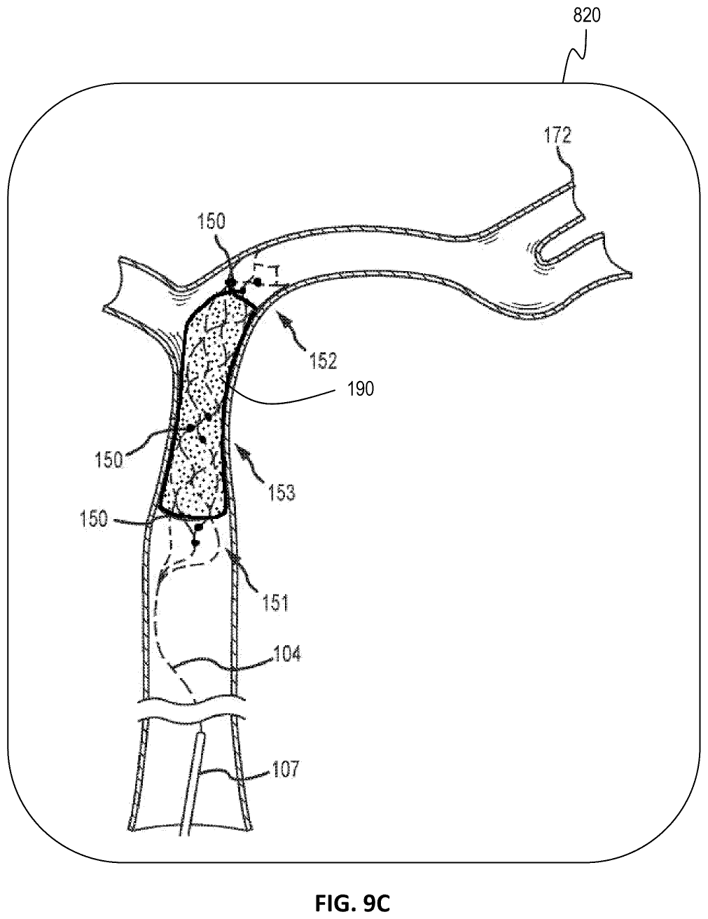

When satisfied by the engagement between the thrombectomy device 102 and the thrombus 165, the user may allow the thrombectomy device 102 to continue expanding into the thrombus 165 (e.g., by further proximally retracting the catheter 107 to fully expose the thrombectomy device 102) and/or proceed to withdraw the thrombectomy device 102 (along with the thrombus 165) from the vessel. As illustrated in FIG. 9A, the thrombectomy device 102 can be withdrawn proximally, along with the thrombus 165, within the vessel. As the thrombectomy device 102 and the thrombus 165 are withdrawn, additional image(s) can be received and analyzed to monitor the engagement between the thrombectomy device 102 and the thrombus 165. During this withdrawal process, the desired engagement between the thrombectomy device 102 and the thrombus 165 may again not necessarily be confirmable by a user directly viewing the imaging, because the thrombus 165 itself may not be visualized under imaging (e.g., fluoroscopic imaging). Accordingly, in some variations the method can include receiving image data indicative of relative positions of markers on the thrombectomy device 102 as it is being withdrawn. FIG. 9B shows an illustrative schematic of an image 810 depicting relative positions of markers on the thrombectomy device 102 as it and the thrombus are being withdrawn (the outline of the thrombectomy device 102 is depicted in broken line for reference, and may or may not be visible in the image 810 itself). Similar to that described above, one or more of such images can be inputted into a trained machine learning algorithm of the prediction module 70 (and the feature extraction module 60, if present), which can be configured to analyze the images 810 to predict at least one characteristic of the thrombus 165 based on the markers on the thrombectomy device.

A predicted characteristic of the thrombus 165 as the thrombus 165 is being withdrawn can be provided to the user such as on a user interface device. In some variations, as shown in FIG. 9C, the predicted characteristic of the thrombus 165 can include a predicted boundary of the thrombus 165 as it is being withdrawn, and the predicted boundary of the thrombus can be displayed overlaid with the image(s) under which the thrombectomy procedure is being performed, in a combined or overlay image 820. However, in some variations the predicted boundary of the thrombus can be displayed separately from such imaging. In other examples, the at least one predicted characteristic of the thrombus can additionally or alternatively be provided to the user in any suitable manner, such as any of the manners discussed above.

A user can use the predicted characteristic(s) of the thrombus to assess whether there is sufficient or otherwise satisfactory engagement between the thrombectomy device 102 and the thrombus 165 (e.g., to provide a high likelihood of success of removing the thrombus from the vessel on the first pass) while the thrombectomy device 102 and the thrombus 165 are being withdrawn. If not satisfied by the engagement between the thrombectomy device 102, the user can perform corrective or repositioning actions such as adjusting the longitudinal position of the thrombectomy device 102, adjusting the rotational position of the thrombectomy device 102, adjusting the amount of radial expansion (e.g., if the thrombectomy device 102 is expanded via a balloon), adjusting the trajectory of the withdrawal path in the vessel, and/or the like. In some variations, repositioning or adjusting the thrombectomy device 102 can include re-collapsing the thrombectomy device 102 such as by distally advancing the catheter 107, and re-expanding the thrombectomy device 102 after repositioning the thrombectomy device 102 such as by again proximally retracting the catheter 107 to expose the repositioning thrombectomy device 102.

The engagement between the thrombectomy device 102 and the thrombus 165 can be continually or periodically monitored similar to the manner(s) described above, as the thrombectomy device 102 and the thrombus 165 are withdrawn from the vessel. As illustrated in FIG. 10, the thrombectomy device 102 can be withdrawn proximally, along with the thrombus 165, into the guide catheter 164. The guide catheter 164 and other components of the thrombectomy system can then be fully removed from the patient.

While the thrombectomy device 102 described above has been described in the context of use during a thrombectomy or blood flow restoration procedure, the thrombectomy device 102 can also, or alternatively, be used as an implantable member (e.g., stent). For example, the thrombectomy device 102 can be released through the connection 106 at a stenosis, aneurysm, or other appropriate location in a vessel. The thrombectomy device 102 can expand and engage a vessel wall so as to hold the vessel wall open and/or act as an occluding member. While the filament thicknesses, widths, cell sizes, and forces described above can be optimized for a thrombectomy device 102 for flow restoration, these values can also be optimized for a thrombectomy device 102 for use as an implantable member. In some variations the same values can be used for both flow restoration and use as an implantable member.

### CONCLUSION

Although many of the embodiments are described above with respect to systems, devices, and methods for guiding a thrombectomy, the technology is applicable to other applications and/or other approaches, such as other blood flow restoration procedures. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-10.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The disclosed technology is illustrated, for example, according to various examples described below. Various examples of examples of the disclosed technology are described as numbered examples (1, 2, 3, etc.) for convenience. These are provided as examples and do not limit the disclosed technology. It is noted that any of the dependent examples may be combined in any combination, and placed into a respective independent example. The other examples can be presented in a similar manner.

Example 1. A method for guiding removal of a thrombus, the method comprising: receiving image data indicative of positions of marker elements on a thrombectomy device positioned proximate the thrombus in a patient; and predicting at least one characteristic of the thrombus by applying a trained machine learning algorithm to the image data.

Example 2. The method of claim 1, wherein the machine learning algorithm analyzes three-dimensional positions of the marker elements.

Example 3. The method of claim 1 or 2, wherein the machine learning algorithm analyzes the position of a marker element at a given timepoint.

Example 4. The method of any one of claims 1-3, wherein the machine learning algorithm analyzes a change in position of a marker element across a period of time.

Example 5. The method of any one of claims 1-4, wherein the machine learning algorithm analyzes the relative positions of multiple marker elements at a given timepoint.

Example 6. The method of any one of claims 1-5, wherein the machine learning algorithm analyzes a change in the relative positions of multiple marker elements across a period of time.

Example 7. The method of any one of claims 1-6, wherein predicting at least one characteristic of the thrombus comprises predicting a boundary of the thrombus.

Example 8. The method of claim 7, wherein predicting at least one characteristic of the thrombus comprises predicting the boundary of the thrombus during expansion of the thrombectomy device in the patient.

Example 9. The method of claim 7 or 8, wherein predicting at least one characteristic of the thrombus comprises predicting the boundary of the thrombus after expansion of the thrombectomy device in the patient.

Example 10. The method of any one of claims 7-9, wherein predicting at least one characteristic of the thrombus comprises predicting the boundary of the thrombus during withdrawal of the thrombectomy device from the patient.

Example 11. The method of any one of claims 1-10, wherein predicting at least one characteristic of the thrombus comprises predicting a material characteristic of the thrombus.

Example 12. The method of claim 11, wherein the material characteristic of the thrombus comprises stiffness of the thrombus.

Example 13. The method of any one of claims 1-12, wherein the machine learning algorithm analyzes a change in relative positions of the marker elements.

Example 14. The method of any one of claims 1-13, further comprising providing the predicted characteristic of the thrombus to a user.

Example 15. The method of claim 14, wherein providing the predicted characteristic of the thrombus comprises displaying the predicted characteristic of the thrombus on a display screen.

Example 16. The method of any one of claims 1-15, wherein the thrombectomy device comprises a stent retriever.

Example 17. The method of any one of claims 1-16, wherein the marker elements are distributed around a circumference of the thrombectomy device.

Example 18. The method of any one of claims 1-17, wherein the marker elements are distributed along an axial length of the thrombectomy device.

Example 19. The method of any one of claims 1-18, wherein the marker elements comprise radiopaque elements.

Example 20. A system comprising: one or more processors; and a memory operably coupled to the one or more processors and storing instructions that, when executed by the one or more processors, cause the computing system to perform operations comprising the method of any one of claims 1-19.

Example 21. The system of example 20, wherein one or more processors and the memory are part of an angiography imaging system.

Example 22. A non-transitory computer-readable storage medium comprising instructions that, when executed by one or more processors of a computing system, cause the computing system to perform operations comprising the method of any one of claims 1-19.

Example 23. A method of treatment, comprising: receiving at least one predicted characteristic of the thrombus predicted by the method of any one of claims 1-19.

Example 24. The method of claim 23, wherein the at least one predicted characteristic of the thrombus comprises a boundary of the thrombus during deployment of the thrombectomy device, wherein the method further comprises adjusting position of the thrombectomy device based at least in part on the predicted boundary of the thrombus during deployment of the thrombectomy device.

Example 25. The method of claim 23 or 24, wherein the at least one predicted characteristic of the thrombus comprises a boundary of the thrombus after deployment of the thrombectomy device, wherein the method further comprises confirming engagement of the thrombectomy device with the thrombus based at least in part on the predicted boundary of the thrombus after deployment of the thrombectomy device.

Example 26. The method of any one of claims 23-25, wherein the at least one predicted characteristic of the thrombus comprises a boundary of the thrombus during withdrawal of the thrombectomy device from the patient, wherein the method further comprises adjusting a technique of the withdrawal of the thrombectomy device based at least in part on the predicted boundary of the thrombus during withdrawal of the thrombectomy device.

1. A method for guiding removal of a thrombus, the method comprising:
receiving image data indicative of positions of marker elements on a thrombectomy device positioned proximate the thrombus in a patient; and
predicting at least one characteristic of the thrombus by applying a trained machine learning algorithm to the image data.

2. The method of claim 1, wherein the machine learning algorithm analyzes three-dimensional positions of the marker elements.

3. The method of claim 1, wherein the machine learning algorithm analyzes the position of a marker element at a given timepoint.

4. The method of claim 1, wherein the machine learning algorithm analyzes a change in position of a marker element across a period of time.

5. The method of claim 1, wherein the machine learning algorithm analyzes the relative positions of multiple marker elements at a given timepoint.

6. The method of claim 1, wherein the machine learning algorithm analyzes a change in the relative positions of multiple marker elements across a period of time.

7. The method of claim 1, wherein predicting at least one characteristic of the thrombus comprises predicting a boundary of the thrombus.

8. The method of claim 7, wherein predicting at least one characteristic of the thrombus comprises predicting the boundary of the thrombus during expansion of the thrombectomy device in the patient.

9. The method of claim 7, wherein predicting at least one characteristic of the thrombus comprises predicting the boundary of the thrombus after expansion of the thrombectomy device in the patient.

10. The method of claim 7, wherein predicting at least one characteristic of the thrombus comprises predicting the boundary of the thrombus during withdrawal of the thrombectomy device from the patient.

11. The method of claim 1, wherein predicting at least one characteristic of the thrombus comprises predicting a material characteristic of the thrombus.

12. The method of claim 11, wherein the material characteristic of the thrombus comprises stiffness of the thrombus.

13. The method of claim 1, further comprising providing the predicted characteristic of the thrombus to a user.

14. The method of claim 13, wherein providing the predicted characteristic of the thrombus comprises displaying the predicted characteristic of the thrombus on a display screen.

15. The method of claim 1, wherein the thrombectomy device comprises a stent retriever.

16. The method of claim 1, wherein the marker elements are distributed around a circumference of the thrombectomy device.

17. The method of claim 1, wherein the marker elements are distributed along an axial length of the thrombectomy device.

18. The method of claim 1, wherein the marker elements comprise radiopaque elements.

19. A system comprising:
one or more processors; and
a memory operably coupled to the one or more processors and storing instructions that, when executed by the one or more processors, cause the computing system to perform operations comprising the method of claim 1.

20. A non-transitory computer-readable storage medium comprising instructions that, when executed by one or more processors of a computing system, cause the computing system to perform operations comprising the method of claim 1.
